(19) United States
(12) Patent Application Publication
(10) Pub. No.: US 2025/0375117 A1
(43) Pub. Date: Dec. 11, 2025

(54) SYSTEM AND METHOD FOR DIGITAL AUSCULTATION

(71) Applicant: Riva Health, Inc., Burlingame, CA (US)

(72) Inventors: Andres Cardona, Burlingame, CA (US); Tuhin Sinha, Burlingame, CA (US)

(73) Assignee: Rival Health, Inc., Burlingame, CA (US)

(21) Appl. No.: 19/231,960

(22) Filed: Jun. 9, 2025

Related U.S. Application Data
(60) Provisional application No. 63/657,298, filed on Jun. 7, 2024.

(51) Int. Cl. A61B 5/024; A61B 5/022; A61B 7/04

(52) U.S. Cl. CPC A61B 5/02422; A61B 5/02233; A61B 7/04

(57) ABSTRACT

The system can include: a sphygmomanometer and a stethoscope. The method can include: collecting data; processing the data; and determining a cardiovascular parameter value. In variants, the system and/or method can function to automatically determine a cardiovascular parameter value for a user from auscultation data (e.g., without manually listening to the auscultation data). Additionally or alternatively, the system and/or method can function to validate a cardiovascular parameter device (e.g., a user device).

### CROSS-REFERENCE TO RELATED APPLICATIONS

This application claims the benefit of U.S. Provisional Application No. 63/657,298 filed 7 Jun. 2024, which is incorporated in its entirety by this reference.

### TECHNICAL FIELD

This invention relates generally to the auscultation field, and more specifically to a new and useful system and method in the auscultation field.

### BRIEF DESCRIPTION OF THE SEVERAL VIEWS OF THE DRAWINGS

FIG. 1 is a schematic representation of a variant of the system.

FIG. 2 is a schematic representation of a variant of the method.

FIG. 3 depicts an example of the system.

FIG. 4 is a schematic representation of an example of the method.

FIG. 5 depicts an illustrative example of processing data.

FIG. 6 depicts a specific example of filtering potential heartbeats.

FIG. 7A depicts an example of evaluating pressure data.

FIG. 7B depicts an example of evaluating audio data.

### DETAILED DESCRIPTION OF THE INVENTION

The following description of the embodiments of the invention is not intended to limit the invention to these embodiments, but rather to enable any person skilled in the art to make and use this invention.

### 1. Overview

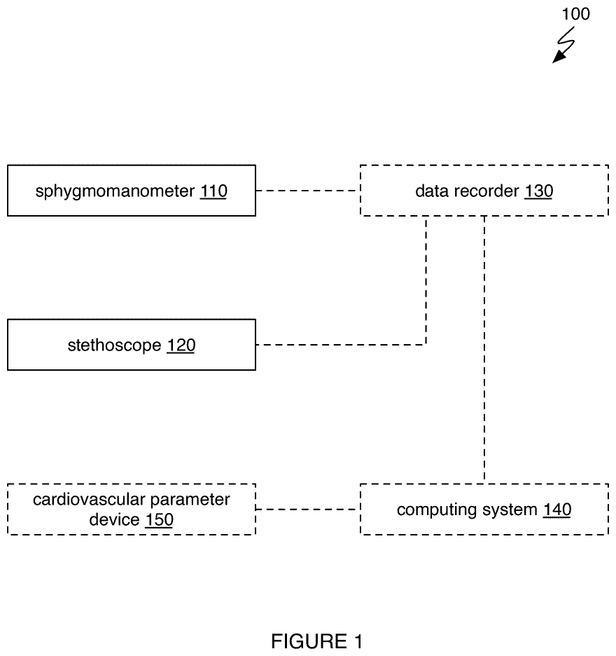

As shown in FIG. 1, the system 100 can include: a sphygmomanometer 110 and a stethoscope 120. However, the system can additionally or alternatively include any other suitable components.

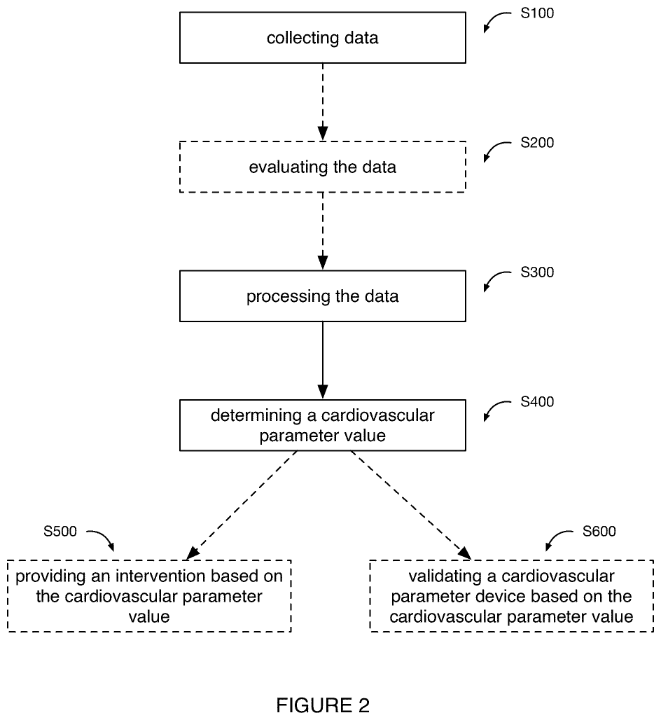

As shown in FIG. 2, the method can include: collecting data S100; processing the data S300; and determining a cardiovascular parameter value S400. However, the method can additionally or alternatively include any other suitable steps.

In variants, the system and/or method can function to automatically determine a cardiovascular parameter value for a user (e.g., patient) from auscultation data (e.g., without manually listening to the auscultation data). The system and/or method can optionally function to validate a cardiovascular parameter device (e.g., a user device).

### 2. Technical Advantages

Variants of the technology can confer one or more advantages over conventional technologies.

Variants of the technology can enable auscultation without manually listening to the arterial blood flow sounds (e.g., without manually listening for sounds indicating times corresponding to systolic and diastolic blood pressure). For example, variants of the technology can provide automatic, digital auscultation with an error (relative to ground truth, manual auscultation) of less than 5 mmHG (e.g., less than 1 mmHG, less than 0.5 mmHG, less than 0.1 mmHG, less than 0.05 mmHG, etc.). In a specific example, variants of the technology can automatically determine a cardiovascular parameter value (e.g., systolic blood pressure, diastolic blood pressure, etc.) using pressure data from a sphygmomanometer and audio data from a stethoscope, without an expert manually identifying heartbeats (e.g., via visual analysis of the audio data and/or listening to the audio data).

However, further advantages can be provided by the system and method disclosed herein.

### 3. System

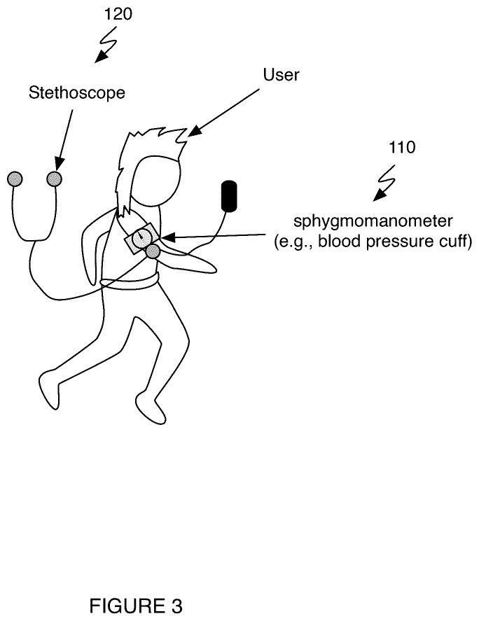

As shown in FIG. 1, the system 100 can include: a sphygmomanometer 110 and a stethoscope 120. The system can optionally include a data recorder 130, a computing system 140, a cardiovascular parameter device 150, and/or any other suitable components. An example of the system is shown in FIG. 3.

The sphygmomanometer 110 (e.g., blood pressure cuff) functions to apply pressure to a body part of a user (e.g., patient) and to measure pressure data. The sphygmomanometer 110 can contact (e.g., touch), be mounted to (e.g., using adhesive, using glue, applying pressure, manually held, etc.), and/or otherwise be coupled to the user. The sphygmomanometer 110 is preferably coupled to the user at a limb or appendage of the user. For example, the sphygmomanometer 110 can be coupled to an arm region (e.g., upper arm, wrist, etc.) of the user. However, the sphygmomanometer 110 can alternatively be coupled to the user at any other body part. The sphygmomanometer 110 is preferably automatic, but can be manual and/or otherwise be operable. However, the sphygmomanometer 110 can be otherwise configured.

The stethoscope 120 functions to measure audio data (e.g., an audio signal) containing arterial blood flow sounds. For example, the stethoscope 120 can be a digital stethoscope. The stethoscope 120 can contact (e.g., touch), be mounted to (e.g., using adhesive, using glue, applying pressure, manually held, etc.), and/or otherwise be coupled to the user. The stethoscope 120 is preferably coupled to the same limb or appendage of the user as the sphygmomanometer 110. For example, the sphygmomanometer 110 and the stethoscope 120 can be mounted to an arm region of the user (e.g., an upper arm region of a user). In a specific example, the sphygmomanometer 110 measures pressure data at the arm region of the user, and the stethoscope 120 measures audio data at the arm region (the same arm region). However, the stethoscope 120 can be otherwise coupled to the user. In a specific example, the stethoscope 120 measures audio data contemporaneously with the sphygmomanometer 110 measuring pressure data. However, the stethoscope 120 can be otherwise configured.

The system can optionally include a data recorder 130, which can function to record pressure data from the sphygmomanometer 110 (e.g., the instantaneous pressure as the sphygmomanometer 110 is inflated and deflated), audio data from the stethoscope 120, and/or any suitable data. For example, the data recorder 130 can include a manometer, a pressure sensor, pressure recorder, and/or any other pressure recording system. In a specific example, the data recorder 130 is configured to simultaneously record pressure data (from the sphygmomanometer 110) and audio data (from the stethoscope 120). However, the data recorder 130 can be otherwise configured.

The system can optionally include a cardiovascular parameter device 150. The cardiovascular parameter device 150 is preferably an unvalidated cardiovascular parameter device, but can alternatively be validated. The cardiovascular parameter device 150 can contact (e.g., touch), be mounted to (e.g., using adhesive, using glue, applying pressure, manually held, etc.), and/or otherwise be coupled to the user. The cardiovascular parameter device 150 is preferably coupled to a different limb or appendage of the user than the sphygmomanometer 110 and the stethoscope 120, which can be beneficial for avoiding crosstalk, contamination, artifacts, or other effects of having two devices for measuring the same or similar cardiovascular parameter in the same blood flow path. For example, the sphygmomanometer 110 and the stethoscope 120 can be mounted to an arm region of the user (e.g., an upper arm region of a first arm of a user) and the cardiovascular parameter device 150 can be contacting a finger of the user's other arm (e.g., a second arm of the user). However, the cardiovascular parameter device 150 can be otherwise coupled to the user. Examples of cardiovascular parameter device 150s include: blood pressure monitors (e.g., palpatory, auscultatory, oscillometric, CNAP, pulse wave velocity, etc.), plethysmometer (e.g., photoplethysmometers such as a pulse oximeter, user device, dedicated instrument, camera, camcorder, etc.), a user device (e.g., phone, including an image sensor), an image sensor (e.g., camera), contactless blood pressure monitors, and/or any suitable devices. An image sensor can optionally include a torch (e.g., camera flash element, lighting element, LED, other light source, etc.). However, the cardiovascular parameter device 150 can be otherwise configured.

The system can optionally include a user interface. The user interface can receive one or more inputs (e.g., from a user), display one or more outputs (e.g., model outputs), display one or more cardiovascular parameters, and/or otherwise function.

The system can optionally include a computing system 140 (e.g., processing system). The computing system 140 can include one or more: CPUs, GPUs, TPUs, custom FPGA/ASICS, microprocessors, servers, cloud computing, and/or any other suitable components. The computing system 140 can be local (e.g., local to one or more components of the system), remote (e.g., cloud computing server, etc.), distributed, and/or otherwise arranged relative to any other system or module. In an example, the computing system 140 can include non-transitory computer-readable media, storing computer-readable instructions that, when executed by the computing system, cause the computing system to perform all or portions of the method (e.g., all or portions of: collecting data S100, evaluating the data S200, processing the data S300, determining a cardiovascular parameter value S400, providing an intervention based on the cardiovascular parameter value S500, and/or validating a cardiovascular parameter device based on the cardiovascular parameter value S600).

The system can optionally include or use one or more models. The models can use classical or traditional approaches, machine learning approaches, and/or other approaches. The models can use or include regression (e.g., linear regression, non-linear regression, logistic regression, etc.), decision tree, LSA, clustering, association rules, dimensionality reduction (e.g., PCA, t-SNE, LDA, etc.), neural networks (e.g., CNN, DNN, CAN, LSTM, RNN, encoders, decoders, deep learning models, transformers, etc.), foundation models, ensemble methods, optimization methods, classification (e.g., binary classifiers, multiclass classifiers, semantic segmentation models, instance-based segmentation models, etc.), rules, heuristics, equations (e.g., weighted equations, etc.), selection (e.g., from a library), lookups, regularization methods (e.g., ridge regression), Bayesian methods (e.g., Naiive Bayes, Markov, etc.), instance-based methods (e.g., nearest neighbor), kernel methods, support vectors (e.g., SVM, SVC, etc.), statistical methods (e.g., probability), comparison methods (e.g., matching, distance metrics, thresholds, vector comparison, image comparison, pattern matching, etc.), deterministics, genetic programs, feature extractors (e.g., Hough transform), image morphology operators (e.g., Dilation, Erosion, Opening, Closing, etc.), information theoretic approaches such as image entropy (e.g., to identify and/or increase feature signals, in the presence of noise), object detectors, key point extraction, segmentation algorithms (e.g., neural networks, thresholding algorithms, clustering algorithms, etc.), any computer vision method (e.g., CV/ML extraction methods), and/or any other suitable architecture. The models can include (e.g., be constructed using) a set of input layers, output layers, and hidden layers (e.g., connected in series, such as in a feed forward network; connected with a feedback loop between the output and the input, such as in a recurrent neural network; etc.; wherein the layer weights and/or connections can be learned through training); a set of connected convolution layers (e.g., in a CNN); a set of self-attention layers; and/or have any other suitable architecture. The models can include less than 10, tens, hundreds, thousands, tens of thousands, hundreds of thousands, and/or any other number of parameters (e.g., weights, biases, etc.). The models can extract data features (e.g., feature values, feature vectors, high-dimensional features, embeddings in a high-dimensional space with hundreds or thousands of dimensions, human-unintelligible features, etc.) from the input data, and determine the output based on the extracted features. However, the models can otherwise determine the output based on the input data.

Models can be trained, learned, fit, predetermined, and/or can be otherwise determined. The models can be trained or learned using: supervised learning, unsupervised learning, self-supervised learning, semi-supervised learning (e.g., positive-unlabeled learning), reinforcement learning, transfer learning, Bayesian optimization, fitting, interpolation and/or approximation (e.g., using gaussian processes), backpropagation, and/or otherwise generated. The models can be learned or trained on: labeled data (e.g., data labeled with the target label), unlabeled data, positive training sets (e.g., a set of data with true positive labels, negative training sets (e.g., a set of data with true negative labels), and/or any other suitable set of data.

Models can optionally be validated, verified, reinforced, calibrated, or otherwise updated based on newly received, up-to-date measurements; past measurements recorded during the operating session; historic measurements recorded during past operating sessions; or be updated based on any other suitable data.

Models can optionally be run or updated: once; at a predetermined frequency; every time the method is performed; every time an unanticipated measurement value is received; or at any other suitable frequency. Any model can optionally be run or updated: in response to determination of an actual result differing from an expected result; or at any other suitable frequency. Any model can optionally be run or updated concurrently with one or more other models, serially, at varying frequencies, or at any other suitable time.

The system can optionally include or use systems disclosed in U.S. application Ser. No. 18/224,174 filed 20 Jul. 2023, and/or U.S. application Ser. No. 17/688,514 filed 7 Mar. 2022, each of which is incorporated in its entirety by this reference.

However, the system can be otherwise configured.

### 4. Method

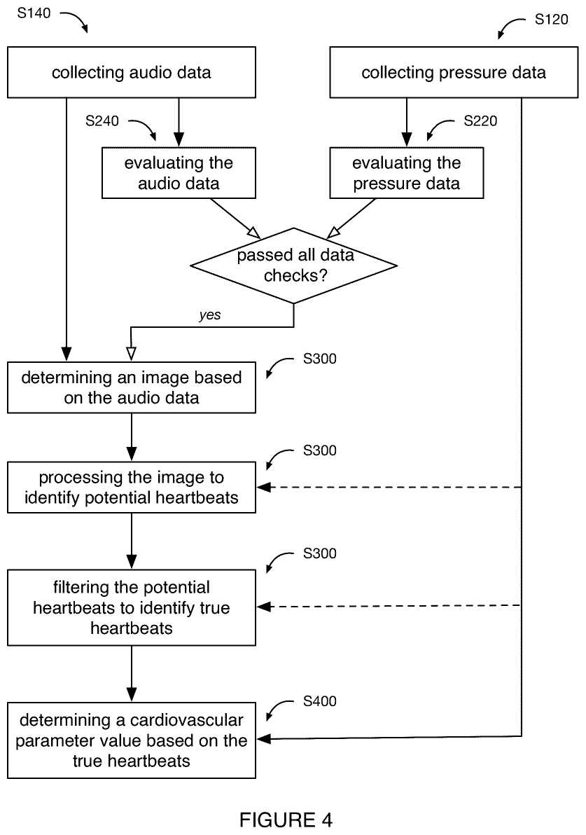

As shown in FIG. 2, the method can include: collecting data S100; processing the data S300; and determining a cardiovascular parameter value S400. The method can optionally include evaluating the data S200, providing an intervention based on the cardiovascular parameter value S500, validating a cardiovascular parameter device based on the cardiovascular parameter value S600, and/or any other suitable steps. An example of the method is shown in FIG. 4.

All or portions of the method can be performed in real time (e.g., responsive to a request), iteratively, concurrently, asynchronously, periodically, and/or at any other suitable time. All or portions of the method can be performed automatically, manually, semi-automatically, and/or otherwise performed. All or portions of the method can be performed by the system 100.

All or portions of the method can be performed in a clinical environment (e.g., a doctor's office, pharmacy, etc.), in a user's home, in a data processing center, distributed between locations (e.g., datasets can be acquired while users are in a clinical environment and processed after the users leave the clinical environment), and/or in any suitable location(s). In a first variant, only S100 is performed while the user is present (e.g., within a clinic). For example, auscultation data (e.g., audio and pressure datasets) can be measured with the user present (e.g., in a clinic, at a first time, etc.), while the systolic and diastolic blood pressure for the user can be determined without the user present (e.g., at a second time after the first time, where the second time can be minutes, hours, days, weeks, etc. after the first time). In a second variant, S100 and S400 are performed while the user is present (e.g., within a clinic). For example, a processing system (e.g., remote from the clinic, local to a device in the clinic, etc.) can process the auscultation data while the user is present, and the systolic and diastolic blood pressure can optionally be presented to the user and/or a clinical provider.

The method can optionally include or use methods disclosed in U.S. application Ser. No. 18/224,174 filed 20 Jul. 2023, and/or U.S. application Ser. No. 17/688,514 filed 7 Mar. 2022, each of which is incorporated in its entirety by this reference.

### 4.1. Collecting Data S100.

Collecting data S100 functions to measure data (e.g., auscultation data, including audio data and pressure data) for a user. S100 can include one or more of: collecting pressure data S120, collecting audio data S140, collecting cardiovascular parameter data S160, collecting any other data (e.g., auxiliary data from the auxiliary device, etc.), and/or any suitable steps. Data can be collected concurrently, asynchronously, and/or at any other time. In an example, S120 and S140 are performed concurrently. In another example, S120, S140, and S160 are all performed concurrently.

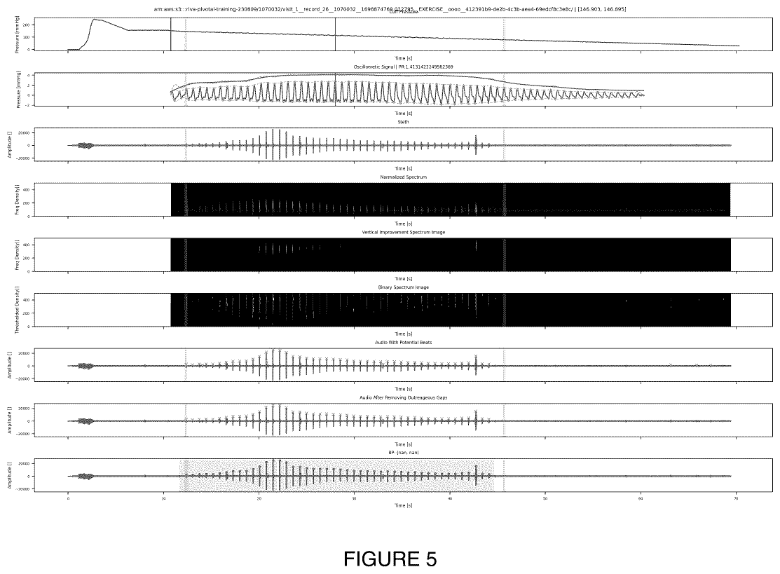

Collecting pressure data S120 functions to acquire pressure measurements spanning systolic and diastolic pressure values for auscultation. The pressure data can be collected using the sphygmomanometer 110 and/or any other suitable device. In an example, over the course of a trial, the sphygmomanometer 110 increases pressure (e.g., at an inflation rate) during an inflation period, and then decreases pressure (e.g., at a deflation rate) during a deflation period. The deflation rate of the sphygmomanometer 110 is preferably at most about 5 mmHg/sec (e.g., 5 mmHg/sec, 4 mmHg/sec, 3 mmHg/sec, 2.5 mmHg/sec, 2 mmHg/sec, 1 mmHg/sec, 0.5 mmHg/sec, 0.1 mmHg/sec, values or ranges therebetween, etc.), which can enable adequate deflation periods to be achieved (e.g., facilitating detection of Korotkoff sounds). However, the deflation rate can be greater than about 5 mmHg/sec. The time of the deflation period is preferably at least about 20 s (e.g., 20 s, 25 s, 30 s, 45 s, 60 s, values or ranges therebetween, etc.), but can be less than about 20 s. The total trial time is preferably at least about 30 s (e.g., 30 s, 35 s, 40 s, 45 s, 60 s, 70 s, 90 s, 120 s, values or ranges therebetween, etc.), but can be less than about 30 s. An illustrative example of pressure data is shown in FIG. 5. In an example, the audio data and the pressure data are measured during all or portions of the inflation period and during all or portions of the deflation period. In a first specific example, the pressure data can include pressure data during the inflation period and the deflation period. In a second specific example, the pressure data can include pressure data during the deflation period (e.g., only the deflation period). However, the pressure data can be otherwise collected.

Collecting audio data S140 functions to acquire audio signals containing arterial blood flow sounds for auscultation. The audio data can be collected using the stethoscope 120 and/or any other suitable device. In a specific example, the audio data is collected contemporaneously with the pressure data. An illustrative example of audio data is shown in FIG. 5. In a first example, the audio data can include audio data during the inflation period and the deflation period. In a second example, the audio data can include audio data during the deflation period (e.g., only the deflation period). However, audio data can be otherwise collected.

S100 can optionally include collecting cardiovascular parameter data S160, which functions to acquire data for validation of the cardiovascular parameter device 150. The cardiovascular parameter data can be collected using the cardiovascular parameter device 150 and/or any other suitable device. In a first example, the cardiovascular parameter data can include a plurality of images acquired while a body region of the user is in contact with an image sensor of the cardiovascular parameter device 150. In a second example, the cardiovascular parameter data can include plethysmogram (PG) data. In a specific example, the cardiovascular parameter data can include photoplethysmogram (PPG) data. In an illustrative example, the PPG data can include aggregate (e.g., sum, weighted sum, etc.) luminance across all or a portion of pixels in an image (e.g., video frame) over time. In variants, the PG data can be derived from images (e.g., contact images of a user body region, remote images of a user body region, etc.). However, the cardiovascular parameter data can include any suitable data and/or be otherwise collected.

However, data can be otherwise collected.

The method can optionally include preprocessing the data. The data can be preprocessed after S100, before S200, after S200, and/or at any other time. In examples, preprocessing can include filtering, downsampling, resampling, smoothing, normalizing, transforming, cropping, and/or otherwise processing the data. For example, data can be preprocessed to remove data prior to the start of deflation (e.g., removing pressure data and/or audio data corresponding to the inflation period). However, data can be otherwise preprocessed. As used herein, “data” can optionally refer to preprocessed data.

### 4.2. Evaluating the Data S200.

The method can optionally include evaluating the data S200, which functions to perform one or more data checks to assess whether all or portions of the data can be used for downstream analysis (e.g., S300, S400, etc.). Data checks can include: one or more audio data checks, one or more pressure data checks, one or more cardiovascular parameter data checks, and/or any other data checks.

S200 can be performed after S100 (e.g., after S120, after S140, etc.) and/or at any other time. S200 can include one or more of: evaluating the pressure data S220, evaluating the audio data S240, evaluating the cardiovascular parameter data, and/or evaluating other datasets. In a specific example, S300 is performed (e.g., the audio data is processed) in response to the pressure data passing the pressure data checks and the audio data passing the audio data checks. In another specific example, a recommendation to perform all or a portion of S100 again is provided if the audio data and/or the pressure data does not pass the audio data checks and/or the pressure data checks, respectively.

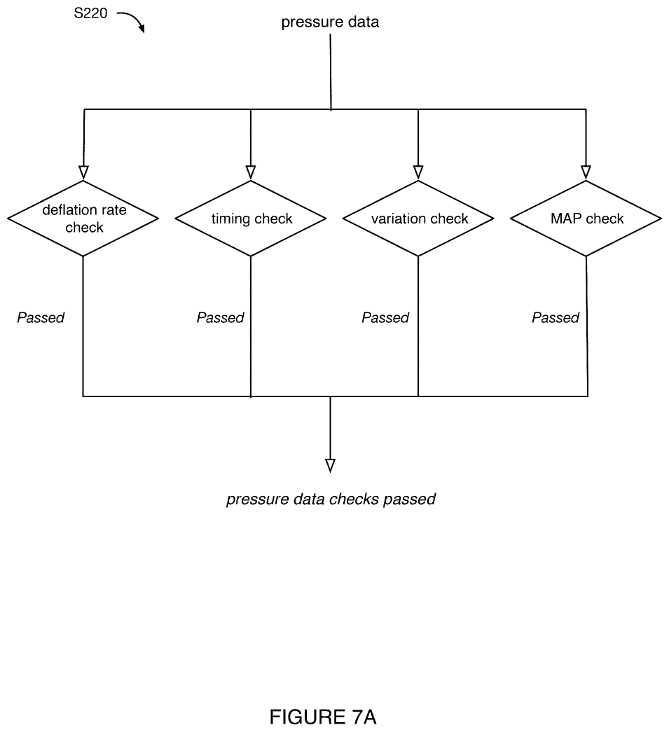

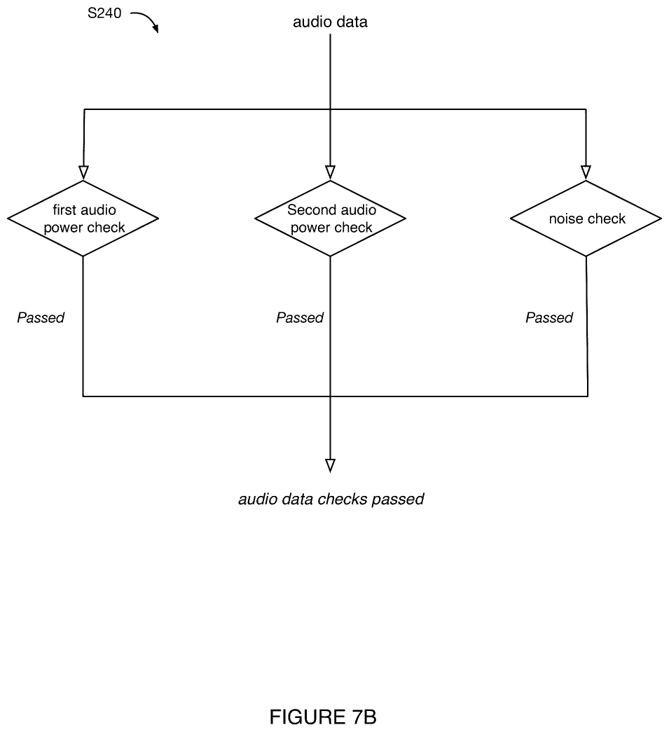

Evaluating the pressure data S220 functions to check the pressure data measured by the sphygmomanometer 110. S220 can include performing one or more pressure data checks on the pressure data. For example, S220 can include performing one or more of: deflation rate check, timing check, variation check, and mean arterial pressure (MAP) check. An example is shown in FIG. 7A. However, any other pressure data checks can be performed. In a specific example, the pressure data passes evaluation if all pressure data checks are passed.

The deflation rate check (e.g., sphygmomanometer deflation rate check) can function to evaluate the rate of change of the pressure data (e.g., the deflation rate of the sphygmomanometer 110). For example, the deflation rate check can include: determining the deflation rate (e.g., the negative rate of change of the pressure data) and evaluating the deflation rate based on a target deflation rate (e.g., comparing the deflation rate to the target deflation rate, comparing the deflation rate to one or more threshold values, etc.). In an example, the deflation rate check is passed when the deflation rate is within a threshold error of a target deflation rate. In a specific example, the target deflation rate can be between 1.5 mmHg/sec-2.5 mmHg/sec or any range or value therebetween (e.g., 2 mmHg/sec), but can alternatively be less than 1.5 mmHg/sec or greater than 2.5 mmHg/sec. In a specific example, the threshold error can be between 0 mmHg/sec-0.5 mmHg/sec or any range or value therebetween (e.g., 0.1 mmHg/sec-0.2 mmHg/sec, 0.15 mmHg/sec, etc.), but can alternatively be greater than 0.5 mmHg/sec. In an illustrative example, the deflation rate check verifies that the deflation rate is between 2.15 mmHg/sec and 1.85 mmHg/sec. As used herein, a positive deflation rate indicates decreasing pressure (e.g., a negative rate of change in the pressure data). However, the deflation rate check can be otherwise performed.

The timing check (e.g., sphygmomanometer deflation timing check) can function to evaluate the timing of deflation of the sphygmomanometer 110 (e.g., relative to the timing of the overall trial). For example, this check can ensure the pressure data captures data spanning from systolic blood pressure to diastolic blood pressure and, optionally, an additional buffer period after the time corresponding to diastolic blood pressure. In a first variant, the timing check includes determining the deflation start time in the pressure data corresponding to the start of the sphygmomanometer deflation (e.g., relative to the start of the trial), and comparing the deflation start time to a threshold. For example, the timing check is passed when the deflation start time is less than or equal to the threshold. In a specific example, the threshold can be between 10 s-40 s or any range or value therebetween (e.g., 15 s-25 s, 20 s, etc.), but can alternatively be less than 10 s or greater than 40 s. In a second variant, the timing check includes determining the total deflation time in the pressure data (e.g., the deflation period, from deflation start to deflation end), and comparing the total deflation time to a threshold. For example, the timing check is passed when the total deflation start time is greater than or equal to the threshold. In a specific example, the threshold can be between 30 s-100 s or any range or value therebetween (e.g., 40 s, 50 s, etc.), but can alternatively be less than 30 s or greater than 100 s. However, the timing check can be otherwise performed.

The variation check (e.g., pressure data linearity check) can function to evaluate the linearity of the pressure data. For example, the variation check can include: determining a coefficient of determination (r²) for the pressure data and comparing the coefficient of determination to a threshold coefficient of determination. For example, the variation check is passed when the coefficient of determination is greater than or equal to the threshold coefficient of determination. The threshold coefficient of determination can be between 0.9-0.99 or any range or value therebetween (e.g., 0.995), but can alternatively be less than 0.9 or greater than 0.999. However, the variation check can include other statistical analyses and/or be otherwise performed.

The mean arterial pressure (MAP) check can function to evaluate the timing of the MAP within the pressure data. For example, the MAP check can include: determining the MAP time (e.g., the time in the pressure data corresponding to the MAP) based on the pressure data and evaluating the MAP time. In a specific example, the MAP check is passed when the MAP time is within a threshold value of the middle of the deflation period of the pressure data. The threshold value can be between 5 s-25 s or any range or value therebetween (e.g., 108-20 s, 15 s, etc.), but can alternatively be less than 5 s or greater than 25 s. In an example, determining the MAP time can include: filtering to the pressure data (e.g., using a low-pass filter, to detrend the pressure data), determining an upper envelope and a lower envelope of the filtered pressure data, determining an amplitude dataset by subtracting the lower envelope from the upper envelope, filtering the amplitude dataset (e.g., using a low-pass filter), and determining the time of the maximum of the filtered amplitude dataset (e.g., where the time of the maximum of the amplitude dataset is the MAP time). However, the MAP check can be otherwise performed.

However, the pressure data can be otherwise evaluated.

Evaluating the audio data S240 functions to check the audio data (e.g., audio signal) measured by the stethoscope 120. S240 can include performing one or more audio data checks on the audio data. For example, S240 can include performing one or more of: a power check and a noise check. An example is shown in FIG. 7B. However, any other audio data checks can be performed. In a specific example, the audio data passes evaluation if all audio data checks (e.g., a first power check, a second power check, a noise check, etc.) are passed.

The power check can function to evaluate the power of the audio signal at the start and end of the deflation period (e.g., verifying that there are no heartbeats recorded in the audio signal at the start and end of the deflation period). In a specific example, the audio data checks can include a first audio power check at a start of the deflation period and a second audio power check at an end of the deflation period. For example, the power check can include, for a segment of the audio data: determining the power (e.g., the total power) of a frequency band of the audio data segment—between a first frequency threshold and a second frequency threshold—and comparing the power to a power threshold. In a specific example, the power check is passed when the power is less than or equal to the power threshold. The first frequency threshold can be 30 Hz-60 Hz or any range or value therebetween (e.g., 50 Hz), but can alternatively be less than 30 Hz or greater than 60 Hz. The second frequency threshold can be 150 Hz-300 Hz or any range or value therebetween (e.g., 200 Hz), but can alternatively be less than 150 Hz or greater than 300 Hz. The audio data segment can be: audio data at the start of the deflation period (e.g., the first 2 s, 5 s, 10 s, 15 s, any range or value therebetween, etc.), audio data at the end of the deflation period (e.g., the last 2 s, 5 s, 10 s, 15 s, any range or value therebetween, etc.), and/or any other audio data segment. The power check can optionally be performed for multiple audio data segments. In a specific example, S240 includes performing a first power check on a first audio data segment and a second power check on a second audio data segment. However, the power check can be otherwise performed.

The noise check can function to evaluate the noise of the audio data. For example, the noise check can include: determining the power (e.g., the total power) of a frequency band of the audio data (e.g., for the deflation period of the audio data)—between a third frequency threshold and a fourth frequency threshold—and comparing the power to a noise threshold. In a specific example, the noise check is passed when the power is less than or equal to the noise threshold. The third frequency threshold can be 300 Hz-700 Hz or any range or value therebetween (e.g., 500 Hz), but can alternatively be less than 300 Hz or greater than 700 Hz. The fourth frequency threshold can be 500 Hz-10,000 Hz or any range or value therebetween (e.g., 1000 Hz), but can alternatively be less than 500 Hz or greater than 10,000 Hz. However, the noise check can be otherwise performed.

However, the audio data can be otherwise evaluated.

However, the data can be otherwise evaluated.

### 4.3. Processing the Data S300.

Processing the data S300 functions to identify heartbeats present in the data for auscultation. S300 can be performed after S100, after S200, and/or at any other time. As used herein, “heartbeat” (e.g., “potential heartbeat” and/or “true heartbeat”) can refer to: the time of the heartbeat, data corresponding to the heartbeat (e.g., image segment, audio data segment, etc.), and/or any other heartbeat information. As used herein, “true heartbeat” (e.g., valid heartbeat, verified heartbeat, real heartbeat, etc.) can be a heartbeat that is assumed (e.g., for determining the cardiovascular parameter value) to correspond to a real heartbeat from the user.

In an example, S300 can include: determining an image based on the audio data, processing the image to identify potential heartbeats in the audio data, and filtering the potential heartbeats to identify true heartbeats in the audio data. An illustrative example is shown in FIG. 5. In a specific example, processing the audio data can include: determining a spectrogram from the audio data; converting the spectrogram into an image; identifying potential heartbeats in the image (e.g., by detecting heartbeat patterns); segmenting the potential heartbeats into a set of segments; and filtering the potential heartbeats to identify true heartbeats (e.g., based on a number of potential heartbeats in each segment of the set of segments, based on a potential pulse rate of potential heartbeats in each segment of the set of segments, etc.).

Determining an image based on the audio data can optionally include: determining a spectrogram from the audio data and converting the spectrogram into an image (e.g., a grayscale image). In a specific example, the window width for the spectrogram can be between 20 ms-100 ms or any range or value therebetween (e.g., 40 ms-60 ms, 50 ms, etc.), but can alternatively be less than 20 ms or greater than 100 ms. In a specific example, the spectrogram windows can have an overlap between 5 ms-40 ms or any range or value therebetween (e.g., at least 5 ms, at least 10 ms, 10 ms-20 ms, 20 ms-30 ms, etc.). However, the spectrogram windows can alternatively not have an overlap. Determining the image can optionally include normalizing (e.g., exponentially normalizing) the spectrogram (e.g., normalizing each window of the spectrogram).

Processing the image functions to identify potential heartbeats in the audio data. Processing the image can optionally include: determining a noise-reduced image and identifying potential heartbeats based on the noise-reduced image.

Determining the noise-reduced image can include reducing noise in the image, identifying features in the image, and/or increasing (e.g., maximizing) features in the image. In a specific example, the features can be features associated with potential heartbeats. In a first variant, determining the noise-reduced image can include transforming each window of the image (e.g., each column of pixels corresponding to a spectrogram window) based on an aggregate intensity (e.g., average intensity, weighted average intensity, etc.) of the window. In a specific example, the pixels in each window of the image are multiplied by the average intensity of the pixels in the respective window. In a second variant, determining a noise-reduced image includes filtering the image. In examples, filtering can include applying a threshold (e.g., global threshold, adaptive threshold, etc.), applying one or more image morphology operators (e.g., for thresholding; Open, Close, etc.), applying information theory operations (e.g., for thresholding; entropy-based information theory operations), adaptive filtering, convolution filtering, and/or any other filtering technique. In a specific example, information theoretic approaches (e.g., image entropy) can be used identify and/or maximize features (e.g., in the presence of noise). In a third variant, determining a noise-reduced image can include using image pyramids (e.g., muti-resolution, multi-scale images). In a specific example, image pyramids can be used to provide robust noise-removal and/or for feature identification (e.g., for identifying potential heartbeats, for identifying features associated with potential heartbeats, etc.). In a fourth variant, determining a noise-reduced image includes performing two or more of the previous variants. For example, determining a noise-reduce image can include performing the first variant and the second variant in series (e.g., filtering the transformed image). As used herein, “image” can refer to a noise-reduced image.

Identifying potential heartbeats can optionally include identifying a set of heartbeat patterns in the image (e.g., processed or unprocessed image). Heartbeat patterns can include: line objects, template heartbeats, and/or any other pattern. In a first variant, identifying potential heartbeats can include detecting a set of line objects (e.g., vertical lines) in the image (e.g., the noise-reduced image). For example, the set of line objects can be detected using the Hough transform. However, any other feature extraction methods, object detection methods, image processing methods and/or models can be used. In a specific example, the set of heartbeat patterns can be line objects that are within a threshold angle of vertical and/or line objects that are greater than or equal to a threshold length. The threshold angle can be between 0°-2° or any range or value therebetween (e.g., 0°, 1°, 2°, etc.), but can alternatively be greater than 2°. The threshold length can be between 5 pixels-1000 pixels or any ranger or value therebetween (e.g., 10 pixels, 20 pixels, 30 pixels, etc.), but can alternatively be less than 5 pixels or greater than 1000 pixels. In a second variant, identifying potential heartbeats can include using pattern recognition and/or pattern matching methods. For example, a template pattern for a heartbeat (specific to the user, specific to the trial, etc.) can be identified in the image (e.g., the noise-reduced image), and the set of potential heartbeats can be the heartbeats that match the template pattern (e.g., based on a bidimensional correlation analysis).

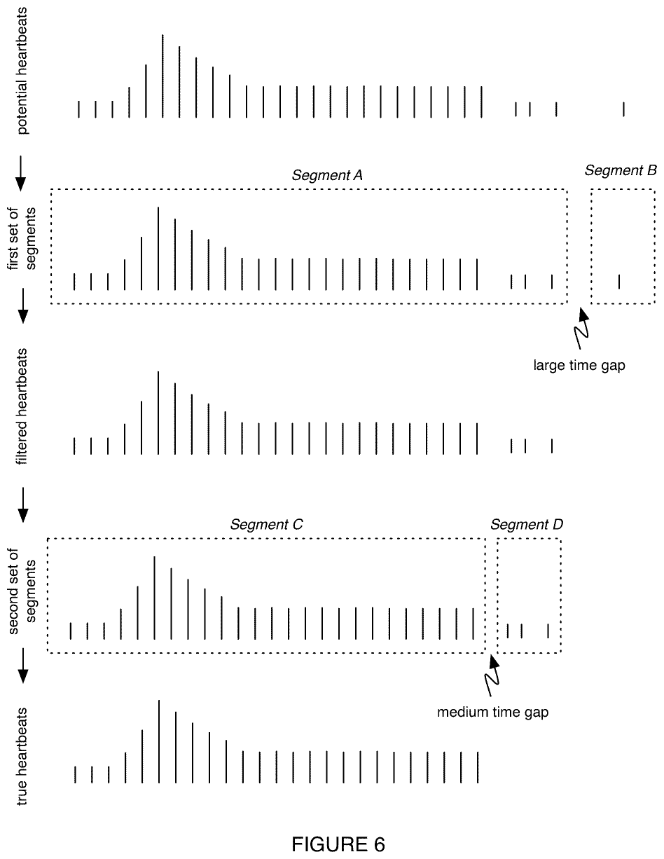

Filtering the potential heartbeats functions to identify a set of true heartbeats in the audio data. A specific example is shown in FIG. 6. The potential heartbeats can optionally be filtered using a set of heuristics. The audio data can optionally be labeled with the set of potential heartbeats (e.g., the audio data can be labeled at the time for each potential heartbeat).

In an example, filtering the potential heartbeats (e.g., performing a filtering operation on the potential heartbeats) can include: segmenting the potential heartbeats into segments (e.g., chunks), and filtering the segments of potential heartbeats (e.g., removing potential heartbeats that are not true heartbeats). The potential heartbeats that remain after filtering one or more times (e.g., after performing one or more filtering operations) can optionally form the set of true heartbeats. The segments are preferably determined based on a pulse rate (e.g., an overall pulse rate), but can be otherwise determined. The pulse rate (e.g., overall pulse rate) is preferably determined based on the pressure data, but can be determined based on the audio data (e.g., based on the potential heartbeats), and/or any other dataset. In an example, segmenting the potential heartbeats can include: identifying time gaps between adjacent potential heartbeats that are greater than a threshold time gap, and segmenting the potential heartbeats such that adjacent segments in the set of segments are separated by the identified time gaps. In a specific example, the threshold time gap can be determined based on the pulse rate (e.g., overall pulse rate).

In a first variant, segmenting the potential heartbeats can include identifying large time gaps (e.g., “outrageous gaps”) between adjacent potential heartbeats, and segmenting the potential heartbeats such that adjacent segments are separated by the large time gaps (e.g., wherein potential heartbeats within a segment are not separated by large time gaps). Large time gaps can be time gaps (e.g., time between adjacent heartbeats) greater than a threshold time gap. In a specific example, the threshold time gap (e.g., a time value) is based on the pulse rate (e.g., overall pulse rate). In a first illustrative example, the threshold time gap can be the inverse of the pulse rate multiplied by a scalar value (e.g., 2, 3, 4, any range or value therebetween, etc.). In a second illustrative example, the threshold time gap can be the pulse rate multiplied by a scalar value (e.g., 2, 3, 4, any range or value therebetween, etc.). In a first example, filtering the segments of potential heartbeats can include potential heartbeats corresponding to each segment with less than a threshold number of potential heartbeats (e.g., 2, 3, 4, any range or value therebetween, etc.). In a second example, filtering the segments of potential heartbeats includes determining a potential pulse rate for each segment (e.g., the median pulse rate based on the timing of the potential heartbeats in the segment), and removing potential heartbeats corresponding to each segment with a potential pulse rate that is outside a target pulse rate range (e.g., greater than a first threshold potential pulse rate and/or less than a second threshold potential pulse rate). In a specific example, the target pulse rate range can be determined based on the pulse rate (e.g., the pulse determined using the pressure data). In a specific example, the target pulse rate range is the pulse rate plus or minus a scalar value (e.g., 0.3-0.6, 0.4-0.5, any range or value therebetween, etc.). In a third example, a combination of the previous examples can be used (e.g., in series).

In a second variant, segmenting the potential heartbeats can include identifying medium time gaps (e.g., “non-outrageous gaps”) between adjacent potential heartbeats, and segmenting the potential heartbeats such that adjacent segments are separated by the medium time gaps (e.g., wherein potential heartbeats within a segment are not separated by medium time gaps). In a specific example, the potential heartbeats can be the potential heartbeats that remain after filtering the potential heartbeats using the first variant (e.g., filtered heartbeats determined using the first variant). Medium time gaps can be time gaps greater than a threshold time gap. In a specific example, the threshold time gap is based on the pulse rate. In a first illustrative example, the threshold time gap can be the inverse of the pulse rate multiplied by a scalar value (e.g., 1.1-2, 1.1-1.5, 1.3, less than the threshold time gap used in the first variant, any range or value therebetween, etc.). In a second illustrative example, the threshold time gap can be the pulse rate multiplied by a scalar value (e.g., 1.1-2, 1.1-1.5, 1.3, less than the threshold time gap used in the first variant, any range or value therebetween, etc.). In an example, the segments of potential heartbeats can be filtered based on the pulse rate and the number of potential heartbeats in each segment. In a specific example, a segment can be removed from the potential heartbeats when the time gap from the segment to an adjacent segment is greater than or equal to the pulse rate multiplied by the number of potential heartbeats in the segment. This can optionally function to filter the potential heartbeats without discarding heartbeats erroneously due to auscultation time gaps between true heartbeats.

In a third variant, a combination of the previous variants can be used (e.g., in series). For example, filtering the potential heartbeats to identify the true heartbeats can include: identifying a first set of segments (e.g., via the first variant), performing a first filtering operation on a first set of segments to identify a first set of filtered heartbeats (e.g., via the first variant), segmenting the first set of filtered heartbeats into a second set of segments (e.g., via the second variant), and performing a second filtering operation on the second set of segments to identify the true heartbeats (e.g., via the second variant). In a specific example, time gaps between adjacent segments of the first set of segments are larger than time gaps between adjacent segments of the second set of segments.

However, the potential heartbeats can be otherwise filtered.

In examples, the processed data can include one or more of: the set of true heartbeats, the time for each true heartbeat in the set of true heartbeats, the audio data labeled with the true heartbeats, and/or any other processed data.

However, the data can be otherwise processed.

### 4.4. Determining a Cardiovascular Parameter Value S400.

Determining a cardiovascular parameter value S400 functions to calculate value(s) for blood pressure (e.g., systolic and diastolic blood pressure) and/or any other cardiovascular parameters. S400 can be performed after S300 and/or at any other time. In an example, the cardiovascular parameter value can be determined based on: the pressure data, the audio data, processed data (e.g., the true heartbeats determined via S300), and/or any other suitable information. In a specific example, the cardiovascular parameter value can be determined based on the true heartbeats (e.g., the time corresponding to one or more true heartbeats) and the pressure data.

Examples of cardiovascular parameters include: blood pressure (e.g., diastolic pressure, systolic pressure, mean arterial pressure (MAP), etc.), arterial stiffness, stroke volume, heart rate, blood volume, pulse transit time, phase of constriction, pulse wave velocity, heart rate variability, blood pressure variability, medication interactions (e.g., impact of vasodilators, vasoconstrictors, etc.), cardiovascular drift, cardiac events (e.g., blood clots, strokes, heart attacks, etc.), cardiac output, cardiac index, systemic vascular resistance, oxygen delivery, oxygen consumption, baroreflex sensitivity, stress, sympathetic/parasympathetic tone, respiratory rate, blood vessel viscosity, venous function, ankle pressure, genital response, venous reflux, temperature sensitivity, and/or any other cardiovascular parameters. In a specific example, the cardiovascular parameter can include one or more of: systolic blood pressure, diastolic blood pressure, and/or mean arterial pressure (MAP).

In a first variant, the cardiovascular parameter value is a systolic pressure value. For example, determining the cardiovascular parameter value can include identifying a time of the first true heartbeat in the set of true heartbeats, and determining the corresponding pressure value at that time based on the pressure data, wherein the corresponding pressure value is the value for systolic pressure.

In a second variant, the cardiovascular parameter value is a diastolic pressure value. For example, determining the cardiovascular parameter value can include identifying a time of the last true heartbeat in the set of true heartbeats, and determining the corresponding pressure value at that time based on the pressure data, wherein the corresponding pressure value is the value for diastolic pressure.

In a third variant, the cardiovascular parameter value is a MAP value. For example, determining the cardiovascular parameter value can include: determining the MAP based on a segment of the pressure data spanning from the time of the first true heartbeat to the last true heartbeat in the set of true heartbeats. In a specific example, determining the MAP can include: filtering to the pressure data segment (e.g., using a low-pass filter, to detrend the pressure data), determining an upper envelope and a lower envelope of the filtered pressure data segment, determining an amplitude dataset by subtracting the lower envelope from the upper envelope, filtering the amplitude dataset (e.g., using a low-pass filter), determining the time of the maximum of the filtered amplitude dataset (the MAP time), and finding the corresponding pressure value at the MAP time in the pressure data, wherein the corresponding pressure value is the value for MAP.

In a fourth variant, a combination of two or more of the previous variants can be used (e.g., to determine values for systolic blood pressure and diastolic blood pressure; to determine values for systolic blood pressure, diastolic blood pressure, and MAP, etc.).

However, one or more cardiovascular parameter values can be otherwise determined.

The method can optionally include storing the cardiovascular parameter value, presenting the cardiovascular parameter value, analyzing the cardiovascular parameter value, providing a recommendation based on the cardiovascular parameter value (e.g., to perform another trial, to consult a physician, etc.), and/or perform any other methods using the cardiovascular parameter value. In specific example, analyzing the cardiovascular parameter value can include: evaluating whether the user has a condition (e.g., hypertension, hypotension, etc.), comparing the cardiovascular parameter value to one or more previous cardiovascular parameter values, and/or performing any other analyses.

### 4.5. Providing an Intervention Based on the Cardiovascular Parameter Value S500.

The method can optionally include providing an intervention (e.g., a therapeutic intervention) based on the cardiovascular parameter value S500. In a specific example, the intervention can be automatically determined and/or automatically provided to the user based on the cardiovascular parameter value. In a specific example, the intervention can function to change the cardiovascular parameter value.

Providing an intervention (e.g., a therapeutic intervention) can include one or more of: automatically modulating medication provision, automatically adjusting an environmental aspect of the user, providing a medical recommendation, facilitating telemedicine digital communications between a user and another entity, and/or any suitable therapy approach. Therapy provision is preferably partially or fully implemented at a remote server, but can be implemented at any suitable component. In variants, providing an intervention can include transmitting instructions to a mobile computing device, the instructions prompting the mobile computing device to instruct a secondary mobile computing device to apply the therapy. In a specific example, intervention provision can include generating, at a remote server, instructions for automatically adjusting an environmental aspect of the user; transmitting the instructions to a mobile computing device (e.g., a smartphone connected to a home network of the user) the instructions prompting the mobile computing device to wirelessly communicate with a secondary device to adjust the environmental aspect (e.g., a television connected to the home network of the user). However, providing an intervention can be performed in any suitable manner.

In a first specific example, the intervention can be provided when the cardiovascular parameter value is above a maximum threshold value. The maximum threshold value can be: predetermined, determined based on previous cardiovascular parameter values for the user, determined using a model, determined based on user information (e.g., demographics, height, weight, patient history, etc.), manually set, and/or otherwise determined. In a second specific example, the intervention can be provided when the cardiovascular parameter value is below a minimum threshold value. The minimum threshold value can be: predetermined, determined based on previous cardiovascular parameter values for the user, determined using a model, determined based on user information (e.g., demographics, height, weight, patient history, etc.), manually set, and/or otherwise determined. In a third specific example, the intervention can be determined and/or adjusted based on the cardiovascular parameter value. For example, a treatment recommendation (e.g., blood pressure measurement frequency, medication dose, diet recommendation, exercise recommendation, sleep recommendation, etc.) can be determined and/or adjusted based on the cardiovascular parameter value.

In a first variant, providing an intervention can include automatically modulating medication provision based on the cardiovascular parameter value. In examples, characteristics of medication provision that can be modulated include: dosage level, dosage frequency, type of medication, medication regimen, medication information, prescription renewal, prescription retrieval, and/or any other suitable medication provision characteristic. Modulation of medication provision can include providing a notification regarding the modulation, automatically communicating with another entity (e.g., renewing a prescription with a pharmacy, contacting a care provider regarding the medication, etc.), and/or any suitable action. In a specific example, providing an intervention can be implemented using automatic medication dispensing apparatus (e.g., a wirelessly-connected medication dispenser), such that this variant of intervention provision can include providing commands from the computing system 140 to the medication dispenser based upon the cardiovascular parameter value.

In a second variant, providing an intervention can include automatically adjusting an environmental aspect of the user based on the cardiovascular parameter value. Adjusting an environmental aspect can include: selecting an environmental aspect to adjust from at least one of lighting audio, and temperature; determining a degree of adjustment (e.g., how much lighting, audio, or temperature to adjust), a timing of adjustment (e.g., automatically adjusting in response to generating an analysis of a cardiovascular parameter, scheduling an adjustment for a particular time or frequency, etc.), and/or any suitable characteristic. A lighting environmental aspect can be the lighting of a mobile computing device of the user (e.g., the mobile computing device used in capturing the time series of image data from which a cardiovascular parameter is determined), a connected lightbulb (e.g., a smart lightbulb connected on the same network as a smartphone of a user), and/or any other suitable lighting component. An audio environmental aspect can be an audio of a mobile computing device (e.g., automatically controlling a mobile computing device to play a selected audio tone or musical sample, modifying the volume setting of a mobile computing device, etc.), a connected audio output device (e.g., a speaker, a television, a secondary mobile computing device, etc.), and/or any suitable device. A temperature environmental aspect can be controlled through a temperature control device (e.g., a connected thermometer, a connected air conditioning and/or heating system, etc.).

In a third variant, providing an intervention can include providing a medical recommendation based on the cardiovascular parameter value. A medical recommendation can be provided to one or more of: a user (e.g., for the user to implement themselves), a care provider, a guardian, and/or any suitable entity. A medical recommendation can include a recommendation to perform a specific action (e.g., to take a walk, to rest, to think positive thoughts, to seek medical attention, to perform all or parts of the method again, etc.), to stop performing a specific action, to take a medication, to communicate with other entity, and/or any suitable activity. The medical recommendation is preferably provided at the mobile computing device associated with the entity to be notified, but can be provided at any suitable device.

In a fourth variant, providing an intervention can include facilitating a digital communication between a user and another entity. A digital communication is preferably enabled between a user and a care provider, but can be enabled between a user and a guardian and/or any relevant entity. A digital communication is preferably enabled through an application (e.g., a phone calling application, a text messaging application, etc.), executing on a mobile computing device associated with a user, but such digital communication can be facilitated through any suitable venue. Facilitating a digital communication between a user and another entity can include: providing the cardiovascular parameter value and/or an analysis of the cardiovascular parameter value to one or more of the user and the other entity, guiding the user and/or the other entity through review of the analysis and/or generation of a treatment based on the analysis, and/or any suitable action.

However, an intervention can be otherwise provided.

### 4.6. Validating a Cardiovascular Parameter Device Based on the Cardiovascular Parameter Value S600.

The method can optionally include validating a cardiovascular parameter device based on the cardiovascular parameter value S600. For example, the cardiovascular parameter device 150 can be an unvalidated cardiovascular parameter device (e.g., as described above). In an example, a second cardiovascular parameter value can be determined using the cardiovascular parameter device 150, and the second cardiovascular parameter value can be compared to the cardiovascular parameter value (determined via S400) to validate the cardiovascular parameter device 150. In a specific example, the second cardiovascular parameter value can be determined using optical data (e.g., PPG data) collected by the cardiovascular parameter device 150 (e.g., user device such as a phone). For example, the cardiovascular parameter device 150 can be validated using methods disclosed in U.S. application Ser. No. 18/224,174 filed 20 Jul. 2023, and/or U.S. application Ser. No. 17/688,514 filed 7 Mar. 2022, each of which is incorporated in its entirety by this reference (e.g., wherein cardiovascular parameters disclosed in the references can correspond to cardiovascular parameter values).

However, the cardiovascular parameter device 150 can be otherwise validated.

### 5. Specific Examples

A numbered list of specific examples of the technology described herein are provided below. A person of skill in the art will recognize that the scope of the technology is not limited to and/or by these specific examples.

Specific Example 1. A system, comprising: a sphygmomanometer configured to measure pressure data at an arm region of a user; a stethoscope configured to measure audio data at the arm region of the user; a data recorder configured to simultaneously record the pressure data from the sphygmomanometer and the audio data from the stethoscope; and a computing system comprising non-transitory computer-readable media, storing computer-readable instructions that, when executed by the computing system, cause the computing system to: determine a spectrogram from the audio data; convert the spectrogram into an image; identify potential heartbeats by detecting heartbeat patterns in the image; segment the potential heartbeats into a set of segments; filter the potential heartbeats to identify true heartbeats, wherein the potential heartbeats are filtered based on at least one of: a number of potential heartbeats in each segment of the set of segments or a potential pulse rate of potential heartbeats in each segment of the set of segments; and determine a cardiovascular parameter value based on the true heartbeats and the pressure data.

Specific Example 2. The system of Specific Example 1, wherein segmenting the potential heartbeats comprises: identifying time gaps between adjacent potential heartbeats that are greater than a threshold time gap, and segmenting the potential heartbeats such that adjacent segments in the set of segments are separated by the identified time gaps.

Specific Example 3. The system of Specific Example 2, wherein the threshold time gap is determined based on an overall pulse rate, the overall pulse rate determined based on the pressure data.

Specific Example 4. The system of any of Specific Examples 1-3, wherein filtering the potential heartbeats to identify the true heartbeats comprises: performing a first filtering operation on the set of segments to identify a first set of filtered heartbeats, segmenting the first set of filtered heartbeats into a second set of segments, and performing a second filtering operation on the second set of segments to identify the true heartbeats.

Specific Example 5. The system of Specific Example 4, wherein time gaps between adjacent segments of the set of segments are larger than time gaps between adjacent segments of the second set of segments.

Specific Example 6. The system of any of Specific Examples 1-5, wherein filtering the potential heartbeats comprises: determining a number of potential heartbeats in each segment of the set of segments, and removing potential heartbeats corresponding to each segment with a number of potential heartbeats that is less than a threshold number.

Specific Example 7. The system of any of Specific Examples 1-6, wherein filtering the potential heartbeats comprises: determining a potential pulse rate for each segment of the set of segments, and removing potential heartbeats corresponding to each segment with a potential pulse rate that is outside a target pulse rate range.

Specific Example 8. The system of any of Specific Examples 1-7, wherein the heartbeat patterns comprise line objects that are within a threshold angle of vertical and are greater than a threshold length.

Specific Example 9. The system of any of Specific Examples 1-8, further comprising a cardiovascular parameter device coupled to the user at a second arm region of the user, wherein the cardiovascular parameter value is used to validate the cardiovascular parameter device.

Specific Example 10. The system of any of Specific Examples 1-9, wherein the cardiovascular parameter value comprises at least one of: a systolic blood pressure value, a diastolic blood pressure value, or a mean arterial pressure value.

Specific Example 11. A method, comprising: measuring pressure data at an arm region of a user using a sphygmomanometer; measuring audio data at the arm region of the user using a stethoscope; processing the audio data, comprising: determining a spectrogram from the audio data; converting the spectrogram into an image; identifying potential heartbeats in the image; segmenting the potential heartbeats into a set of segments; and filtering the potential heartbeats to identify true heartbeats, wherein the potential heartbeats are filtered based on at least one of: a number of potential heartbeats in each segment of the set of segments or a pulse rate of potential heartbeats in each segment of the set of segments; and determining a cardiovascular parameter value based on the true heartbeats and the pressure data.

Specific Example 12. The method of Specific Example 11, wherein the image comprises a set of windows corresponding to windows of the spectrogram, the method further comprising determined a noise-reduced image by transforming each window of the image based on an aggregate intensity of the window, wherein the potential heartbeats are identified using the noise-reduced image.

Specific Example 13. The method of any of Specific Examples 11-12, further comprising providing a therapeutic intervention based on the cardiovascular parameter value.

Specific Example 14. The method of any of Specific Examples 11-13, wherein segmenting the potential heartbeats comprises: identifying time gaps between adjacent potential heartbeats that are greater than a threshold time gap, the threshold time gap determined based on an overall pulse rate, the overall pulse rate determined based on the pressure data; and segmenting the potential heartbeats such that adjacent segments in the set of segments are separated by the identified time gaps.

Specific Example 15. The method of any of Specific Examples 11-14, wherein filtering the potential heartbeats to identify the true heartbeats comprises: performing a first filtering operation on the set of segments to identify a first set of filtered heartbeats, segmenting the first set of filtered heartbeats into a second set of segments, and performing a second filtering operation on the second set of segments to identify the true heartbeats.

Specific Example 16. The method of Specific Example 15, wherein time gaps between adjacent segments of the set of segments are larger than time gaps between adjacent segments of the second set of segments.

Specific Example 17. The method of any of Specific Examples 11-16, further comprising: performing a set of pressure data checks on the pressure data and performing a set of audio data checks on the audio data, wherein the audio data is processed in response to the pressure data passing the pressure data checks and the audio data passing the audio data checks.

Specific Example 18. The method of Specific Example 17, wherein the pressure data checks comprise: a sphygmomanometer deflation rate check, a sphygmomanometer deflation timing check, a pressure data linearity check, and a mean arterial pressure check.

Specific Example 19. The method of any of Specific Examples 17-18, wherein the audio data and the pressure data are measured during an inflation period and during a deflation period, wherein the audio data checks comprise: a first audio power check at a start of the deflation period, a second audio power check at an end of the deflation period, and a noise check.

Specific Example 20. The method of any of Specific Examples 11-119, wherein the cardiovascular parameter value comprises at least one of: a systolic blood pressure value, a diastolic blood pressure value, or a mean arterial pressure value.

All references cited herein are incorporated by reference in their entirety, except to the extent that the incorporated material is inconsistent with the express disclosure herein, in which case the language in this disclosure controls.

As used herein, “substantially” or other words of approximation (e.g., “about,” “approximately,” etc.) can be within a predetermined error threshold or tolerance of a metric, component, or other reference (e.g., within +/−0.001%, +/−0.01%, +/−0.1%, +/−1%, +/−2%, +/−5%, +/−10%, +/−15%, +/−20%, +/−30%, any range or value therein, of a reference).

Optional elements, which can be included in some variants but not others, are indicated in broken line in the figures.

Different subsystems and/or modules discussed above can be operated and controlled by the same or different entities. In the latter variants, different subsystems can communicate via: APIs (e.g., using API requests and responses, API keys, etc.), requests, and/or other communication channels. Communications between systems can be encrypted (e.g., using symmetric or asymmetric keys), signed, and/or otherwise authenticated or authorized.

Alternative embodiments implement the above methods and/or processing modules in non-transitory computer-readable media, storing computer-readable instructions that, when executed by a processing system, cause the processing system to perform the method(s) discussed herein. The instructions can be executed by computer-executable components integrated with the computer-readable medium and/or processing system. The computer-readable medium may include any suitable computer readable media such as RAMs, ROMs, flash memory, EEPROMs, optical devices (CD or DVD), hard drives, floppy drives, non-transitory computer readable media, or any suitable device. The computer-executable component can include a computing system and/or processing system (e.g., including one or more collocated or distributed, remote or local processors) connected to the non-transitory computer-readable medium, such as CPUs, GPUs, TPUS, microprocessors, or ASICs, but the instructions can alternatively or additionally be executed by any suitable dedicated hardware device.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

We claim:

1. A system, comprising:
a sphygmomanometer configured to measure pressure data at an arm region of a user;
a stethoscope configured to measure audio data at the arm region of the user;
a data recorder configured to simultaneously record the pressure data from the sphygmomanometer and the audio data from the stethoscope; and
a computing system comprising non-transitory computer-readable media, storing computer-readable instructions that, when executed by the computing system, cause the computing system to:
determine a spectrogram from the audio data;
convert the spectrogram into an image;
identify potential heartbeats by detecting heartbeat patterns in the image;
segment the potential heartbeats into a set of segments;
filter the potential heartbeats to identify true heartbeats, wherein the potential heartbeats are filtered based on at least one of: a number of potential heartbeats in each segment of the set of segments or a potential pulse rate of potential heartbeats in each segment of the set of segments; and
determine a cardiovascular parameter value based on the true heartbeats and the pressure data.

2. The system of claim 1, wherein segmenting the potential heartbeats comprises: identifying time gaps between adjacent potential heartbeats that are greater than a threshold time gap, and segmenting the potential heartbeats such that adjacent segments in the set of segments are separated by the identified time gaps.

3. The system of claim 2, wherein the threshold time gap is determined based on an overall pulse rate, the overall pulse rate determined based on the pressure data.

4. The system of claim 1, wherein filtering the potential heartbeats to identify the true heartbeats comprises: performing a first filtering operation on the set of segments to identify a first set of filtered heartbeats, segmenting the first set of filtered heartbeats into a second set of segments, and performing a second filtering operation on the second set of segments to identify the true heartbeats.

5. The system of claim 4, wherein time gaps between adjacent segments of the set of segments are larger than time gaps between adjacent segments of the second set of segments.

6. The system of claim 1, wherein filtering the potential heartbeats comprises: determining a number of potential heartbeats in each segment of the set of segments, and removing potential heartbeats corresponding to each segment with a number of potential heartbeats that is less than a threshold number.

7. The system of claim 1, wherein filtering the potential heartbeats comprises: determining a potential pulse rate for each segment of the set of segments, and removing potential heartbeats corresponding to each segment with a potential pulse rate that is outside a target pulse rate range.

8. The system of claim 1, wherein the heartbeat patterns comprise line objects that are within a threshold angle of vertical and are greater than a threshold length.

9. The system of claim 1, further comprising a cardiovascular parameter device coupled to the user at a second arm region of the user, wherein the cardiovascular parameter value is used to validate the cardiovascular parameter device.

10. The system of claim 1, wherein the cardiovascular parameter value comprises at least one of: a systolic blood pressure value, a diastolic blood pressure value, or a mean arterial pressure value.

11. A method, comprising:
measuring pressure data at an arm region of a user using a sphygmomanometer;
measuring audio data at the arm region of the user using a stethoscope;
processing the audio data, comprising:
determining a spectrogram from the audio data;
converting the spectrogram into an image;
identifying potential heartbeats in the image;
segmenting the potential heartbeats into a set of segments; and
filtering the potential heartbeats to identify true heartbeats, wherein the potential heartbeats are filtered based on at least one of: a number of potential heartbeats in each segment of the set of segments or a pulse rate of potential heartbeats in each segment of the set of segments; and
determining a cardiovascular parameter value based on the true heartbeats and the pressure data.

12. The method of claim 11, wherein the image comprises a set of windows corresponding to windows of the spectrogram, the method further comprising determined a noise-reduced image by transforming each window of the image based on an aggregate intensity of the window, wherein the potential heartbeats are identified using the noise-reduced image.

13. The method of claim 11, further comprising providing a therapeutic intervention based on the cardiovascular parameter value.

14. The method of claim 11, wherein segmenting the potential heartbeats comprises: identifying time gaps between adjacent potential heartbeats that are greater than a threshold time gap, the threshold time gap determined based on an overall pulse rate, the overall pulse rate determined based on the pressure data; and segmenting the potential heartbeats such that adjacent segments in the set of segments are separated by the identified time gaps.

15. The method of claim 11, wherein filtering the potential heartbeats to identify the true heartbeats comprises: performing a first filtering operation on the set of segments to identify a first set of filtered heartbeats, segmenting the first set of filtered heartbeats into a second set of segments, and performing a second filtering operation on the second set of segments to identify the true heartbeats.

16. The method of claim 15, wherein time gaps between adjacent segments of the set of segments are larger than time gaps between adjacent segments of the second set of segments.

17. The method of claim 11, further comprising: performing a set of pressure data checks on the pressure data and performing a set of audio data checks on the audio data, wherein the audio data is processed in response to the pressure data passing the pressure data checks and the audio data passing the audio data checks.

18. The method of claim 17, wherein the pressure data checks comprise: a sphygmomanometer deflation rate check, a sphygmomanometer deflation timing check, a pressure data linearity check, and a mean arterial pressure check.

19. The method of claim 17, wherein the audio data and the pressure data are measured during an inflation period and during a deflation period, wherein the audio data checks comprise: a first audio power check at a start of the deflation period, a second audio power check at an end of the deflation period, and a noise check.

20. The method of claim 11, wherein the cardiovascular parameter value comprises at least one of: a systolic blood pressure value, a diastolic blood pressure value, or a mean arterial pressure value.
